(19) United States
(12) Patent Application Publication
(10) Pub. No.: US 2023/0201253 A1
(43) Pub. Date: Jun. 29, 2023

(54) SYSTEMS AND METHODS FOR ADMINISTERING HYDRATION COMPOSITIONS

(71) Applicants: Juan Carlos Parodi, Buenos Aires (AR); Samuel Fernandez, Buenos Aires (AR); Scott R. Miller, Arlington, VA (US)

(72) Inventors: Juan Carlos Parodi, Buenos Aires (AR); Samuel Fernandez, Buenos Aires (AR); Scott R. Miller, Arlington, VA (US)

(21) Appl. No.: 18/117,321

(22) Filed: Mar. 3, 2023

Related U.S. Application Data
(62) Division of application No. 16/765,142, filed on May 18, 2020, now abandoned.
(60) Provisional application No. 62/586,858, filed on Nov. 15, 2017.

(51) Int. Cl. A61K 33/00; A23L 33/19; A23L 33/00; A23L 33/175; A23L 33/16; A23L 33/125; A23L 33/15; A61P 3/12; A61B 5/11; A61B 5/00; A61K 9/00; A61K 31/198; A61K 31/405; A61K 31/7004; A61K 38/00; A61K 47/10; A61K 47/36; A61K 47/42

(52) U.S. Cl. CPC A61K 33/00; A23L 33/15; A23L 33/16; A23L 33/19; A23L 33/30; A23L 33/40; A23L 33/125; A23L 33/175; A61B 5/1118; A61B 5/4875; A61B 5/6887; A61B 5/7275; A61K 9/0053; A61K 31/198; A61K 31/405; A61K 31/7004; A61K 38/00; A61K 47/10; A61K 47/36; A61K 47/42; A61P 3/12; A23V 2002/00; A61B 5/026; A61B 5/0082

(57) ABSTRACT

Systems and methods for administering one or more hydration compositions to an individual to improve vascular health are provided. A system comprises a hydration composition delivery element and a sensor operatively coupled to the hydration composition delivery element. The sensor is configured to determine one or more of the intake of fluids, the amount of fluid discharged, the activity level of the patient, or level of hydration of the patient based on the level of fluid in the hydration composition delivery element. The system further includes a monitoring system which, based on a signal received from the sensor, provides an indication of one or more of the need to intake fluid, the identity of a specific hydration composition of a plurality of hydration compositions to be consumed or an amount of a hydration composition to be consumed.

### CROSS REFERENCE TO RELATED APPLICATIONS

This application is a divisional of U.S. Application 16/765,142, filed May 18, 2020, which is a U.S. National Stage Application of International Application No. PCT/US2019/023594, filed Jan. 15, 2019, which claims the benefit of U.S. Provisional Application No. 62/586,858 filed Nov. 15, 2017, the entire disclosures of which are incorporated herein by reference for all purposes.

### TECHNICAL FIELD

The present disclosure relates to systems and methods for improving vascular health in a patient suffering from intermittent claudication or rest pain of the lower extremities. More particularly, the present disclosure is directed towards systems for administering one or more hydration compositions to a patient in need thereof. More particularly the hydration system includes a delivery element having both a sensor and a monitoring system.

### BACKGROUND

Currently, many people of all ages fail to maintain proper hydration throughout a given day. This is a result of many factors, including failing to consume enough liquids and food to sustain hydration, consuming liquids and foods that do not match the hydration needs of the individual, engaging in physical activity and failing to hydrate or hydrating with liquids that do not match the physiological needs and activity of the user, taking in foods and fluids that increase elimination (such as caffeinated drinks and alcohol), as well as other factors that can adversely affect hydration, including aging, disease, injury, other physiological change, side effects from medicines, and environmental factors like changing external temperature and humidity levels.

The majority of the human body is related to hydration: between 55 and 60% of the body’s weight is due to fluid in the form of body water. Further, even when specific physiological systems are examined in more specific detail, such as the vascular system, body water alone is approximately five percent of the intra-vascular fluid. Poor hydration can adversely affect intra-vascular function, tissue perfusion, organ performance, strength, endurance, infection fighting ability, cognitive function and other physiological elements.

In addition, the body is not static in its level of hydration, even if physical activity is minimal. About 1,500 milliliters of water is eliminated daily by the kidneys and about 1,000 ml by perspiration, respiration and feces. Failure to replenish eliminated and consumed fluids may result in multiple adverse events. A change as low as a one percent reduction in overall hydration can lead to head aches, muscle tiredness, sleepiness and other physical limitations. Hydration losses of two percent or more can result in reduced cognitive performance and additional physical limitations. A broad array of physiological functions are tied to proper and consistent hydration, including nutrient transportation, body temperature regulation, toxin and waste elimination, delivery of oxygen at a cellular level, electrical function of a variety of cells, absorption of and transportation of oxygen and many other functions.

Inadequate hydration can also contribute to the advancement of chronic disease and can be a complicating factor with rare diseases, including cystic fibrosis, Bartter Syndrome, and other rare diseases. Research indicates inadequate hydration is a factor in intermittent lower extremity claudication, which can become disabling and can transform into critical limb ischemia with risk of amputation. Life expectancy of patients with critical limb ischemia is significantly decreased compared with the general population indicating the presence of extended atherosclerosis. Other health issues tied to inadequate hydration include chronic kidney disease, among other health issues. Peripheral and other forms of vascular disease may also be adversely affected by inadequate hydration.

Intermittent claudication of the lower extremities is a common condition in the elderly population. Peripheral Artery Disease (PAD) is detected in 18 to 29% of people aged 60 years old and over. An estimated 5 to 10 million people in the United States have PAD, and many are either under diagnosed or may exhibit “silent” disease until complications such as leg ulcers appear.

Intermittent lower extremity claudication is, in general, a benign condition. Sometimes, however, it can become disabling and, even less frequently, transforms into critical limb ischemia with risk of amputation. Life expectancy of patients with Critical Limb Ischemia is significantly decreased compared with the general population indicating the presence of extended atherosclerosis.

The common femoral artery is the most common place of initiation of plaques of atherosclerosis. Main risk factors are age, genetic predisposition, smoking habit, diabetes, dyslipidemia and hypertension. Intermittent claudication is usually managed medically, controlling risk factors, promoting exercises (supervised exercise program), Cilostazol, and antiplatelet drugs. Controlled exercises and medication are often effective and constitute the only treatment for these patients. When disabling claudication persists in spite of treatment or critical limb ischemia takes place, percutaneous treatment if often indicated and provides successful results in a significant number of patients. Plain balloon angioplasty, use of drug eluting balloons, stents, drug eluting stents and covered stents are the resources typically used by interventionists.

Surgery is utilized in selected patients; endarterectomies or bypasses are the techniques available to improve circulation. Endovascular treatments are expensive and lesions recur with certain frequency obligating to apply new costly devices.

Surgical vein or PTFE bypasses are effective and durable but they have some risk in elderly and debilitated patients.

Maintaining adequate hydration is not as simple as merely drinking more water or other fluids. Either under or over-hydrating can create health and performance issues and hydration needs change based on a variety of factors, including conditions specific to the individual, external factors, age, disease, activity levels, and other factors, including what fluid is consumed by the individual at specific points in time. Further, merely adding electrolytes or other supplements is not always beneficial. There are times when the individual needs electrolytes to improve hydration and overall performance, but there are other times when adding electrolytes and other supplements can hurt performance and therefore is contrary to what is needed (including causing hypernatremia). In certain circumstances, mere intake of water, or intake of a drink with a different formulation may be beneficial. However, in other instances, drinking water without supplements can end up causing an outflow or dilution of key nutrients, causing complications such as hyponatremia.

Based on clinical observations, dehydration is a cause of aggravation of ischemia and that proper hydration improves blood perfusion. Accordingly, it would be advantageous to provide for hydration compositions as a part of a hydration system to maintain beneficial hydration over time for optimal performance and health. Additionally, it would be advantageous to provide a delivery system to provide the correct volume and hydration composition needed to achieve the appropriate daily hydration that are tied to precise feedback of hydration levels and overall physiological needs of each individual to support overall health, limit advancement of chronic diseases, increase immune system support, improve performance, and other notable benefits.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure relates to a method for improving vascular health in a patient suffering from intermittent claudication or rest pain of the lower extremities by administering a hydration composition to the patient in an amount sufficient to reduce at least one of: the mean value of temperature in the feet; pain intensity; distance to claudication as determined using a treadmill; time to claudication as determined using a treadmill; or ankle/brachial index.

In another aspect, the present disclosure relates to a hydration system including a plurality of hydration compositions for improving vascular health. The hydration compositions may be formulated for oral or enteral administration and, depending on the health or physical activity of the patient, may include proteins, electrolytes, vitamins, minerals, and a flavoring agent. The plurality of hydration compositions include: a hydration composition to sustain hydration when the individual is at rest or engaged in moderate activity; a hydration composition for re-hydration and sustained hydration during higher performance; and a hydration composition for rapid recovery from high activity.

In another aspect, the present disclosure relates to a system for administering one or more hydration compositions to a patient. The delivery system includes a sensor to determine the intake of fluids, the amount of fluid discharged, the activity level of the patient, and/or level of hydration of the patient and provide a signal back to a monitoring system. Based on information received from the sensor, the monitoring system provides an indication to the patient or a third party (e.g., a clinician) of the need to intake fluid and/or which hydration composition of a plurality of hydration compositions should be consumed and in what amount. The monitoring system may include a smart phone, a patient monitoring system, a nurse’s station, or any other suitable monitoring system.

In another aspect, the present disclosure relates to method of using a delivery system for administering one or more hydration compositions to a patient. The method includes: sensing one or more hydration-indicative parameter of the patient, the one or more hydration-indicative parameter selected from amount of fluid intake, amount of fluid discharge, activity level of the patient, or level of hydration of the patient; generating a signal indicative of the hydration-indicative parameter; transmitting the signal to a monitoring system; and based on the signal received from the sensor, providing an indication to the patient or a third party of: the need to intake fluid; the identity of a specific hydration composition of a plurality of hydration compositions to be consumed; or an amount of a hydration composition to be consumed.

### BRIEF DESCRIPTION OF THE DRAWINGS

The above and other aspects, features, and advantages of the present compositions and associated delivery and monitoring systems will become more apparent in light of the following detailed description when taken in conjunction with the accompanying drawings in which:

FIG. 1A shows an illustrative delivery system with sensor, which can measure the consumption of fluid from the delivery system and communicate the level of fluid consumption;

FIG. 1B shows an illustrative receiving element to track, analyze and communicate information regarding hydration and fluid consumption, including recommended intake volumes, time and compositions;

FIG. 2 is a flowchart describing the steps performed in a process that is in accordance with an exemplary embodiment of a hydration system of the present disclosure;

FIG. 3 is a graphic depicting skin temperature in a patient before treatment using a hydration system in accordance with this disclosure;

FIG. 4 is a graphic depicting skin temperature in a patient after treatment using a hydration system in accordance with this disclosure;

FIG. 5 is a graphic showing the temperature of the dorsal aspect of the first toe before and after treatment;

FIG. 6 is a graphic showing the plantar temperature of the first right toe before and after treatment;

FIG. 7 is a graphic showing subjective pain intensity sensation evaluated before and after treatment;

FIG. 8 is a graphic showing the distance to claudication before and after treatment as determined using a treadmil;

FIG. 9 is a graphic showing the time to claudication before and after treatment as determined using a treadmil; and

FIG. 10 is a graphic showing ankle/brachial index determined before and after treatment.

### DETAILED DESCRIPTION

Particular embodiments of the present hydration system that includes a portfolio of hydration related formulations are described hereinbelow with reference to the accompanying drawings; however, it is to be understood that the disclosed embodiments are merely exemplary of the disclosure and the present system and formulations may be embodied in various forms. One skilled in the art will readily recognize from the following discussion that alternative embodiments of the structures and methods illustrated herein may be employed without departing from the principles of the present disclosure described herein. Therefore, specific hydration compositions and system details disclosed herein are not to be interpreted as limiting, but merely as a basis for the claims and as a representative basis for teaching one skilled in the art to variously employ the concepts of the present disclosure in virtually any appropriately detailed system or hydration composition.

The present disclosure relates to solutions that address the spectrum of hydration needs. In addition, a delivery system and a feedback and compliance system are also disclosed so that the specific hydration needs of the individual can be quickly ascertained and linked to the proper solution to consume to maintain hydration, with monitoring and reminders to enhance compliance and overall results. The determination of which formulation to consume is based on specific feedback provided to the user by a hydration sensor and feedback system, including algorithms for analyzing, projecting and recommending specific intake formulations and timing. Combined together, the formulations and sensors allow the user to match their specific hydration need with the intake of an optimal formula to obtain, maintain and sustain hydration to support desirable physiological, neurological and overall function.

In embodiments, a hydration composition in accordance with this disclosure includes water, proteins, electrolytes, vitamins, minerals, and a flavoring agent to support sustained hydration.

In embodiments, a hydration composition in accordance with this disclosure includes water in an amount from about 80% to about 99.9%, in embodiments from 90% to 100%, in other embodiments from 98% to 99.9%. Suitable water for the present hydration compositions may include natural, spring or prepared water; water that has been distilled, filtered, or otherwise treated, or water with modification of its pH.

In embodiments, a hydration composition in accordance with this disclosure may further include proteins in an amount from about 0.001% to about 20%, in embodiments from 0.001% to 5%, in other embodiments from 0.001% to 1.0%. Proteins help to sustain hydration by modifying oncotic or hydrostatic pressure, thereby keeping water in the circulatory system longer than if a solution was consumed without proteins in the composition. In embodiments, hydration compositions in accordance with this disclosure include albumin, globulins and/or fibrinogen, or other suitable proteins or additives that promote synthesis of albumin by the liver, or other means, to modify oncotic pressure. In embodiments, protein may be added at a concentration of as much as 22 grams per liter for an individual of 70 Kg of weight and 28 grams for a 90 Kg person, or less or more if indicated.

In embodiments, a hydration composition in accordance with this disclosure may include one or more electrolytes in an amount from about 0.001% to about 20%, in embodiments from 0.001% to 5%, in other embodiments from 0.001% to 1.0%. In embodiments, a hydration composition in accordance with this disclosure includes electrolytes such as alkali and alkaline earth salts, including salts of potassium, calcium, magnesium, or sodium such as, for example, sodium chloride, sodium acetate, sodium citrate, sodium bromide, sodium phosphate, potassium acetate, potassium bromide, potassium chloride, potassium citrate, calcium acetate, calcium chloride, calcium phosphate, and magnesium chloride.

In embodiments, a hydration composition in accordance with this disclosure further includes vitamins in an amount from about 0.001% to about 20%, in embodiments from 0.001% to 5%, in other embodiments from 0.001% to 1.0%. Suitable vitamins may include: Vitamin B Complex (B1 (thiamin), B2 (riboflavin), niacin (nicotinic acid), B6 (pyridine), B12 (cyanocobalamin), folic acid, pantothenic acid, biotin (helps the body convert carbohydrates into energy); ascorbic acid (vitamin C—promotes iron absorption); cholecalciferol (vitamin D₃ promotes calcium absorption).

In embodiments, a hydration composition in accordance with this disclosure further includes minerals in an amount from about 0.001% to about 20%, in embodiments from 0.001% to 5%, in other embodiments from 0.001% to 1.0%. Suitable minerals for a hydration composition in accordance with this disclosure include calcium, iron, and any supplements containing combinations of calcium and iron.

A hydration composition in accordance with this disclosure may further include a flavoring agent in an amount from about 0.001% to about 20%, in embodiments from 0.001% to 5%, in other embodiments from 0.001% to 1.0%. Flavoring agents include those flavors known to one of ordinary skill in the art, such as natural flavors, artificial flavors, spices, seasonings, and the like. Specific flavoring agents for use in the sports beverage composition include those flavoring agents that can impart a complementary character flavor to the off notes provided by the added proteins are suitable.

A hydration composition in accordance with this disclosure may further include a beneficial therapeutic ingredient. Beneficial therapeutic ingredients, such as prebiotic fiber (oligofructose, fructose, sprouted mung bean extract and acesulfame K) which promotes vitamin B production and calcium absorption, crystalloids to manage the delivery and absorption of the composition, and other ingredients deemed to be beneficial to hydration and overall circulatory performance. Using these additives to sustain the level of water in the circulatory system includes the added benefit of obtaining balanced hydration for the individual through a lower level of overall fluid consumption by virtue of modifying oncotic pressure or other means to cause the consumed fluids to remain in the circulatory system longer.

In embodiments, a hydration composition in accordance with this disclosure may further include essential amino acids in an amount from about 0.001% to about 20%, in embodiments from 0.001% to 5%, in other embodiments from 0.001% to 1.0%. In embodiments, included essential amino acids include Leucin, Isoleucine, Lysine, Methionine, Valine and Tryptophan. In embodiments, included non-essential amino acids are: Alanine, Proline, Glycine, Glutamine, Glutamic acid, Arginine, Cysteine, Histidine, Serine, Aspartic acid, Asparagine and Tyrosine.

In embodiments, a hydration composition in accordance with this disclosure may further include an ingredient to adjust hydrostatic pressure. In embodiments, glucose or other forms of sugars may be included and pectin, starch or other elements that are able to act as a hydrogel or otherwise sustain the delivery and absorption of one or more of the elements in the formulation, including carbohydrates, amino acids or glucose or other sugars.

Examples of the portfolio of compositions for use with a hydration system in accordance with the present disclosure are described below:
(i) An exemplary hydration composition to sustain hydration when the individual is at rest or engaged in moderate activity is provided. This composition includes albumin or other suitable proteins in an amount of from about .25 mg to about 1.5 mg per kilogram of weight of user weight. This composition includes sodium at a concentration of up to 15 mEq per liter for a hypertensive user or up to 30 mEq per liter for a normotensive user. Potassium in the solution may be added at a concentrate of up to 20 mEq per liter. To improve absorption, a different ratio of albumin or essential amino acids may be substituted for protein or may be included in a reduced or altered mix of protein. Essential amino acids in this composition may be selected from leucin, isoleucine, lysine, methionine, valine and tryptophan. Additional amino acids may be added to this hydration composition, such as, alanine, proline, glycine, glutamine, glutamic acid, arginine, cysteine, histidine, serine, aspartic acid, asparagine and tyrosine.
(ii) An exemplary composition for re-hydration and sustained hydration during higher performance is provided. This composition contains between 0 to 0.5% albumin or other suitable proteins, between 5 and 40 grams of carbohydrates, and electrolytes and /or glucose in a delivery technology for absorption without creating intestinal distress, which may be a hydrogel, a pectin based additive, a starch, a glycerin or other element to sustain and improve absorption of electrolytes and which may include potassium or certain amino acids included in similar levels as are shown in exemplary composition (i) above, and
(iii) a composition for rapid recovery from high activity including 10 to 20 grams of protein and 10 to 30 grams of carbohydrates and similar compositions of other additives as contained in compositions (i) and (ii) above. Vitamins, minerals, and amino acids may also be added to any composition hereunder. In embodiments, variations on these compositions and other elements described herein (including types of fluids, gels, powders and other hydration focused components) may also be part of the system and its constituent drinks. In addition, in certain instances and circumstances, the system may promote and recommend the consumption of water.

The target daily intake for an individual utilizing a hydration system in accordance with this disclosure varies by activity, as well as the compositions of the fluids. However, an individual generally aims to intake between 2,500 and 3,500 milliliters of water per day. The system may be used to deliver the formula orally, enterally, or intravenously.

In certain embodiments, hydration compositions in accordance with this disclosure may be combined with a therapeutic drug having time released delivery mechanisms to sustain the presence of the therapeutic drug and/or the hydration fluid by adjusting oncotic or hydrostatic pressure to effect more beneficial delivery.

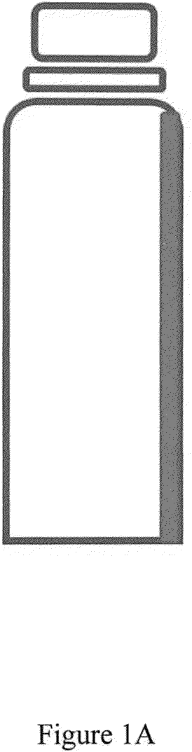

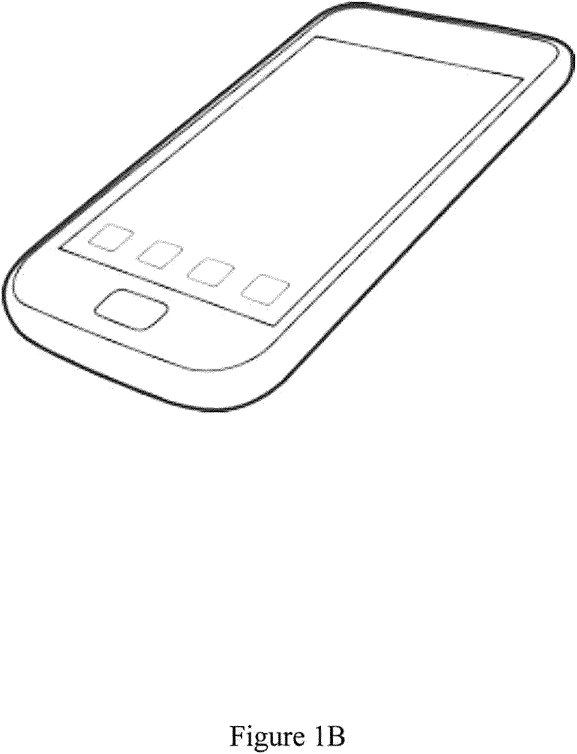

A hydration system according to the present disclosure may further include a delivery system, as well as monitoring and feedback mechanisms. In embodiments, a hydration system in accordance with this disclosure may include a single-use or reusable delivery element, which may be a flexible package, a bottle or other element capability of holding fluid. FIG. 1A illustrates an exemplary hydration system including fluid delivery element 10, and sensor 20 attached thereto. In embodiments, sensor 20 is able to measure the level of fluid in the delivery element and the change in the level of fluid and to communicate it to receiving element 30 of the system, which may generally be any receiver and processor of information to determine fluid consumption and to use to analyze the fluid consumption of the user, such as a patient monitoring system, communication device, tablet, smart phone or other device, as best seen in FIG. 1B. In embodiments, the delivery element may also have a sensor or other element supporting the communication of the change in the level of fluid in the delivery element though interaction with the monitoring system, which may be a patient monitoring system, a smart watch, a smart phone or personal communication device or other element capable of assessing and determining the level of fluid change in the delivery system.

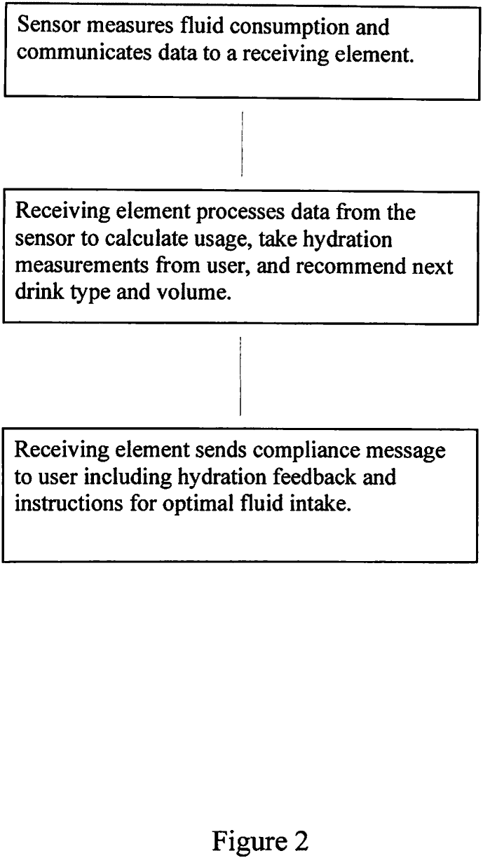

FIG. 2 is a flowchart showing the steps performed in a process in accordance with an exemplary embodiment of a monitoring and feedback mechanism of a hydration system of the present disclosure. In embodiments, sensor 20 may be scannable by a reading device, such as a smart phone, or have its own ability to transmit a signal to receiving element 30 of the system. Once a signal is received by receiving element 30, the signal may be used to send reminders to the user or to other individuals to maintain compliance with a hydration regimen. The signal may also be used to calculate the intake of fluid and the type of fluid in order to measure consumption and this information may be used with proprietary algorithms to project the next intake time of fluid and the type of fluid to intake. The system may interact with the user to determine the current and next level of activity levels to recommend a suitable hydration approach by level of activity and by time of day. This interaction may include questions and answers with the users, as well as monitoring of the user to determine the best approach. The monitoring may include motion sensors to determine activity, a pedometer, a heart rate and/or blood pressure monitor or other means to obtain user information and use to formulate the hydration approach.

In addition, receiving element 30 may measure the individual’s hydration through one or more capabilities, such as an ultrasound scanner focused on assessing hydration through imaging of vascular elements of the individual, a saliva based hydration sensor, an infrared reader of tissue hydration that can be used to scan or assess hydration through tissue (such as an appendage) or other suitable hydration sensing and measurement element. This hydration level input is also provided to receiving element 30 or may be a part of the receiving element 30 (such as a sensor in a smart phone). The information from the hydration sensor is used to recommend the intake of one or more fluids of varying compositions based on the level of hydration and the user’s projected activity level.

In embodiments, the monitoring and feedback mechanisms of the hydration system may be configured to determine various parameters, including, the individual’s current level of hydration, the individual’s level of electrolytes, the individual’s level of blood sugar, the individual’s level of tissue perfusion, oxygen saturation, pulse, heart rate, cardiac output and any other physiological parameters related to overall hydration and health. The monitoring system in embodiments includes a means to provide a specific recommendation as to which formulation of the current disclosure and volume is recommended for consumption by the individual, as well as the volume and recommended rate of consumption. The formulations that may be recommended and linked to the monitoring system include, in embodiments, a formulation version that increases electrolytes; in embodiments, a version that increases blood sugar; in embodiments, a version that adds fluid alone; in embodiments, a version that adds fluid with modest sodium; in embodiments, a version that adds fluid and a protein or other ingredients to sustain, increase or otherwise modify oncotic pressure; in embodiments, amino acids. The monitoring system also includes the ability to track and register the type and amount of hydration consumed in order to analyze, assess and provide further algorithms and other feedback mechanisms for the user. This includes, for example, identifying times of the day when hydration may decline, when blood sugar level may decline, or when other physiological parameters may change that tie to hydration.

The plurality of the formulation compositions may be between 0.001 percent and 25 percent of the weight of the liquid application when combined with a liter of fluid to hydrate. Specific components of the formulation may be targeted at certain amounts linked to the weight or body mass of the user and the feedback monitoring system in embodiments, may contain data as to fluid consumption relative to individual characteristics (such as weight and/or body mass and/or activity levels), specific inputs as to age and various disease conditions, and learning elements to track usage and employ machine learning to adjust and refine fluid consumption usage and activities level of continue to refine and then recommend total hydration levels of a period of time, such as on a daily and weekly basis, and more specific recommendations and reminders on a more frequent basis, including hourly and other interim periods.

The end objectives of the compositions may vary. For example, one composition may be focused on sustaining hydration by keeping water in the circulatory system longer. An additional composition may be focused on facilitating more rapid absorption of ingredients important to higher energy activities, such as a composition with more electrolytes and carbohydrates. An additional composition may be focused more on facilitating recovering from high energy activities and then transitioning to a sustained hydration intake composition. All of this is linked together through the direction and feedback provided by the hydration assessment, feedback and monitoring system.

The feedback sensor may assess hydration and other parameters by any feasible approach, including a saliva test, an infrared measurement system through the skin, an optical sensor, an ultrasound sensor, or any other means to determine hydration and other related physiological parameters.

The sensor and other aspects of the feedback mechanism may be present in one or more device, including as part of a wrist watch, a wrist band, a ring, a smart phone or other electronic device, a finger probe, a diagnostic strip that can be placed in the mouth or licked and then submitted into a reader; a saliva sensor that can be placed on any device that can be brought to the mouth; an optical sensor that can be embedded in eyewear; an ultrasound sensor that can be placed over target areas of the body (including the inferior or superior vena cava or other vasculature) to determine hydration related changes including changes in vessel size and volume; and any other sensor that can be portable and used to determine hydration and related parameters, including sensors that may be disposable, wearable or of other portable means.

In embodiments, the sensor and monitoring device may include the ability to provide remote signaling to a third party for monitoring and compliance. In embodiments, the monitoring device and/or the sensor may include the ability to deliver advertisements and/or reminders for compliance to the device related to the user’s physiological parameters, or to a different electronic device (such as a smart phone or an alternative mobile device)

The sensor and monitoring device may be part of one device or multiple, separate devices. The sensor and monitoring devices could be mobile or tied into or linked to a stationary patient monitory system, communication device, tablet, smart phone or other receiver and processor of information.

### EXAMPLES

Dehydration often occurs in elderly population. Incidence of peripheral artery disease in patients over 60 years of age ranges between 18 and 29%. It was hypothesized that dehydration aggravates symptoms of PAD. Patients with disabling claudication or rest pain resistant to standard medical therapy were instructed to drink between 2,500 to 3000 milliliters of water according to their body mass. 36 patients affected of disabling intermittent claudication or rest pain of the lower extremities were treated. Proper hydration was achieved in 35 patients, all of them improved their status related to lower extremity ischemia. Increase of ankle/brachial index was observed in most of them (p: 0.0001) and time and distance to claudication using a treadmill test occurred in all patients who were properly hydrated (p>0.0001). Thirty-five of the thirty-six patients drank 2,500 milliliters of fluid or more. The non-compliant patient did not have any variation of symptoms, skin temperature, ankle/brachial index or time and distance to claudication.

The outcome after 6 weeks from the initiation of the protocol is described below. Patients without a past history of Congestive Heart Failure or renal insufficiency were included in the following example without interrupting exercises they were performing or medication they were receiving. Ninety percent of the patients were receiving aspirin and 100% of them Statins and Cilostazol at least for 5 months before starting the protocol. (See Table 2 below.)

All patients belonged to category IIb of Fontaine and grade 1 category 2 and 3 of Rutherford. Only patients, who have failed to improve their intermittent claudication and were suffering from disabling claudication or rest pain after standard medical treatment, were included.

Protocol: Complete physical examination and laboratory work were performed. Ankle-brachial pressures were recorded.

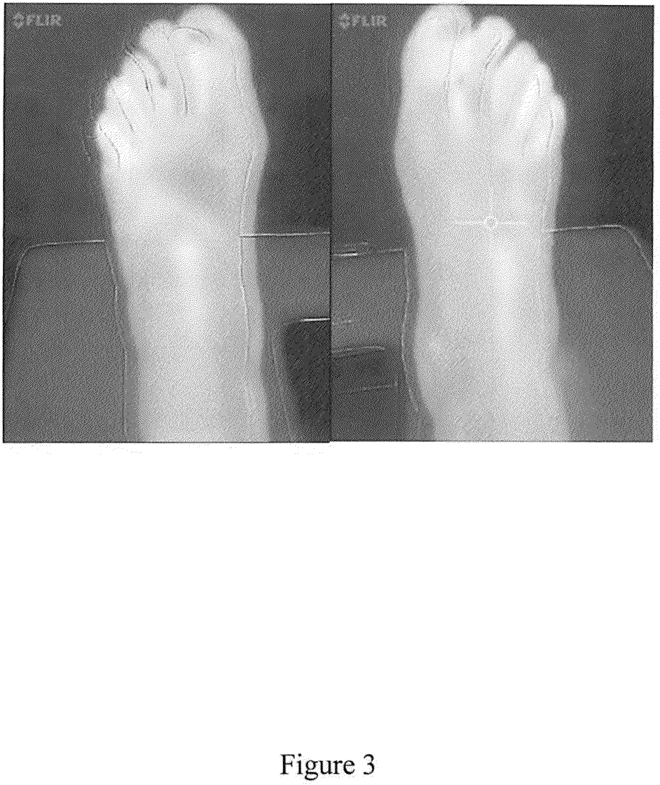

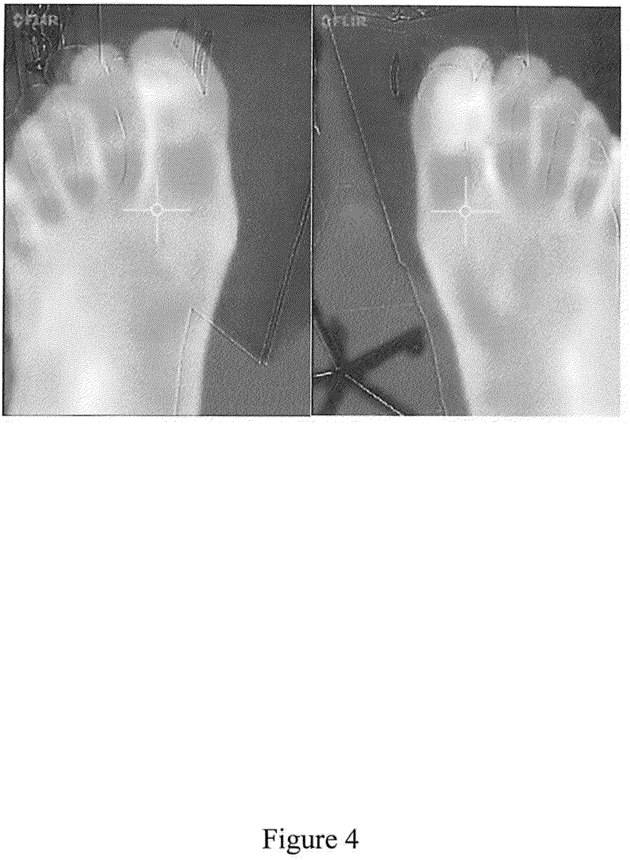

1) Graphics of skin temperature were obtained in both feet using a Flir Thermal Camera (Flir Systems Inc.) in a room at 24° C. (See FIGS. 3 and 4)

2) Time and distance to claudication using a treadmill at 3 miles per hour was recorded.

3) Subjective classification of pain intensity from 1 to 10 was documented. (0= no pain, 10=intense pain)

4) Skin color - including the absence and/or reduction in abnormal variations in skin color, skin tone and skin elasticity.

Duplex of the abdominal artery, iliac arteries and infra-inguinal vessels was performed in every patient. Patients were followed weekly and underwent regular blood tests. Patients were recommended to drink 3,000 milliliters of water a day; fluids like tea and milk were counted in addition to water. This recommendation was the part of the protocol and it was carefully controlled.

Statistical analysis: software utilized: SPSS 20 Qualitative variables were expressed in percentages. Quantitative variables were analyzed with the test of Shapiro-Wilk. Continuous variables were expressed as median, 95% confidence intervals and interquartile intervals. Wilcoxon Signed Rank Test was also utilized P<0.05 was considered significant.

### Case 1

An 84 years old lady complaining of 10 step intermittent claudication of the lower extremities, abdominal angina that produced a 15 Kg weight loss, and an abnormal renal function with a creatinine of 3.1 mg per deciliter.

The patient had a past history of hypertension and was a heavy smoker (more than 30 cigarettes a day). The patient was having intermittent claudication for years but in the last four months it became disabling. The patient suffered 2 acute myocardial infarctions; coronary angiogram showed non-correctable coronary artery disease.

On physical exam patient had no palpable pulses. A CT Angiogram depicted a right subclavian tight stenosis, both carotid arteries and vertebral arteries had moderate disease. The left subclavian artery was chronically occluded. The thoracic aorta had diffuse “coral reef” lesions with occlusion at the level of the diaphragm that extended to the distal aorta, which was open as well as the iliac and infra-inguinal arteries. The patient refused surgeons, and surgeons, due to her high risk, were hesitant to encourage her to undergo surgical treatment (aortic endarterectomy).

A decision was made to offer a less extensive surgery consisting of right subclavian stenting and an axillobifemoral bypass. The procedure was performed successfully and the patient improved significantly. The patient was able to walk without limits, the intestinal angina subsided and renal function improved. Open wide collaterals produced an increased perfusion of the bowel and kidneys. The patient was discharged in three days. The patient did well for three months when she called to the hospital telling the physician on call, that she was having the same symptoms she had before surgery. The bypass was open, but the patient referred to physicians that she had diarrhea for the last four days. The patient was admitted, stool cultures performed and patient was rehydrated with precaution considering her heart condition. The second day the patient asked for lunch and ate it without having abdominal pain. The patient was able to walk 50 meters without pain. After four days the patient was discharged and asked to drink 2.5 liters of fluids a day. 14 months after the last admission, the patient remained asymptomatic walking, without pain, more than 4 blocks.

### Case 2

A 76-year-old patient came to the consult with right foot rest pain, pallor and coldness up to the knee. Patient had 2 bypasses and two stents placed on the right side after thrombosing a popliteal aneurysm and all the procedures failed. Rest pain started one week before the consult and his claudication became disabling one month before. The patient was drinking less than 700 milliliters a day of fluids. No suitable distal vessel to perform a new revascularization was found. Before proceeding to amputation, proper hydration was achieved. After two days, pain subsided and the skin of the right foot was pink. Three liters of fluid a day was recommended and the patient was discharged. After three months, the patient walked three blocks without pain. After seven months, the patient walked four kilometers without pain.

### 36 Patient Trial

A demographic analysis of the 36 patients included in the trial and the medications they were taking are presented in the following Tables 1 and 2, respectively.

TABLE 1

Demographics (n 36)

| Age (Median and inter-quartile interval) | 76 (60-79) years |
|---|---|
| Males | 66.66% |
| Hypertension | 80% |
| Diabetes | 33.3% |
| Smoking Habit | 50% |
| Hyperlipidemia | 50% |
| Obesity | 33.3% |
| Chronic renal insufficiency | 20% |
| Chronic Atrial Fibrillation | 6.6% |
| Previous stroke | 16.6% |
| Past history of acute myocardial infarct. | 6.6% |

TABLE 2

Medications of patients included in the protocol: Percentage of patients receiving the specific medication (n 36)

| | |
|---|---|
| Aspirin | 90% |
| Clopidogrel | 10% |
| Coumadin | 6.6% |
| Antihypertensive drugs | 63.6% |
| Beta-blockers | 31.8% |
| Calcium Antagonists | 18.2% |
| Statins | 100% |
| Cilostazol | 100% |
| Metformin | 23.3% |
| Insulin | 20% |
| Morphine | 22.7% |
| Pregabalin | 13.6% |

Time and distance to claudication using the treadmill, cutaneous temperature, ankle-brachial pressure and subjective pain sensation improved significantly in patients who drank more than 2.5 liters of fluids a day (35 of the 36 patients). The only patient who did not improve drank less than 2 liters of fluid a day.

Fluid Intake: Before starting the protocol, patients were drinking 1000 milliliters as median (1000-1000), the mean value was 1095.83 (+/- 521.58) milliliters. During the protocol fluid intake increased to a median of 2,500 milliliters (2000-3000) and mean of 2,750 (+/- 712.13) milliliters, p:0.0001.

Changes in skin temperature of the feet: A significant increase in skin temperature was found after 6 weeks of hydration. Median temperature of the dorsum of the right foot before initiating the protocol was 29.95 (27.60-31.15) °C. After hydration: 30.35 (28.12-31.60) °C. p: 0.02. Median temperature of the plantar aspect of the right foot: 28.70 (27-31) °C. Median temperature after 6 weeks: 29 (27.70-31) °C. p:0.011. Median temperature of the dorsum of the left foot before the protocol: 29.40° C. (27.72-31.30). Median temperature of the dorsum of the left foot after 6 weeks: 30.20 (27.70-31.70) °C. p: 0.009. Median temperature of the plantar aspect of the left foot before hydration: 28.40 (26.82 -30) °C. Median temperature of the plantar aspect of the left foot after hydration: 29.20 (26.70 - 30.92) °C. p: 0,030.

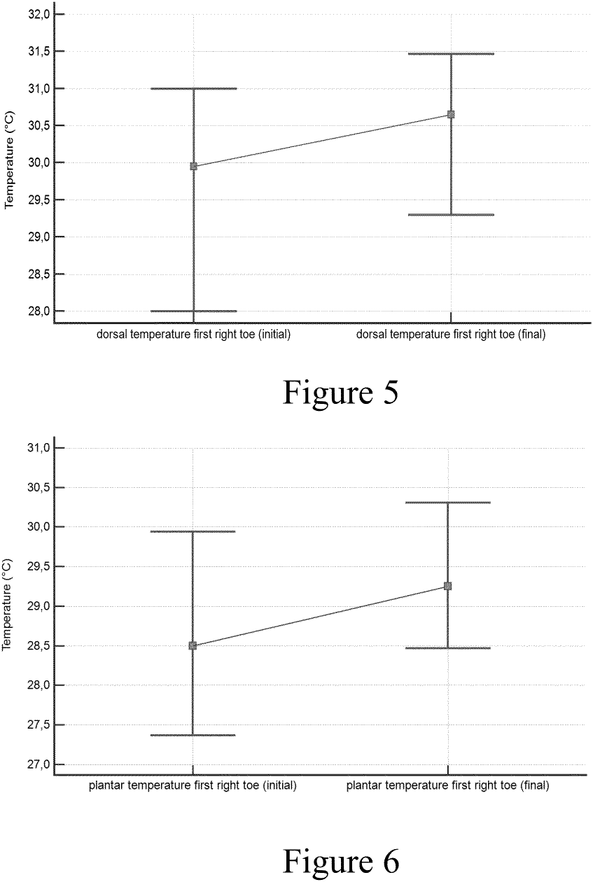

FIG. 5 shows the temperature of the dorsal aspect of the first toe before and after treatment (using median values and 95% confidence intervals). The initial Median dorsal temperature of the first right toe before treatment was 29.95 IC (28 to 31) and the final Median dorsal temperature of the first right toe after treatment was 30.65 IC (29.300 to 31.468).

FIG. 6 shows the plantar temperature of the first right toe before and after treatment. Before treatment, the initial Median was 28.50 CI (27.366 to 29.939) and the final Median plantar temperature of the first right toe after treatment was 29.25 CI (28.464 to 30.302). The difference was statistically significant. Wilcoxon test (paired samples). Two-tailed probability p:0.0347.

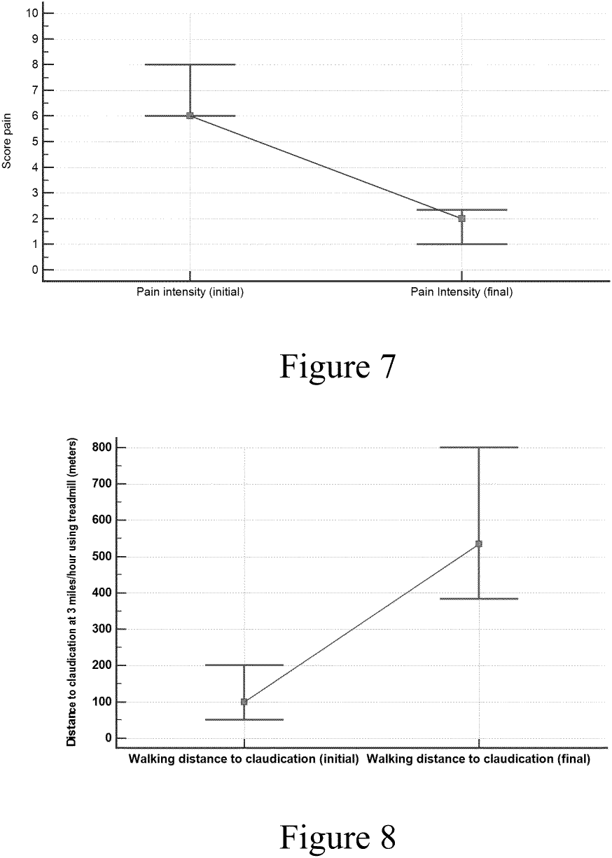

Pain sensation: Subjective pain intensity sensation was evaluated before and after treatment (using median values and 95% confidence intervals). The pain intensity (initial) Median was 6 CI (6 to 8) and the pain intensity (final) Median was 2 CI (1 to 2.341). The difference was statistically significant Wilcoxon test (paired samples) Two-tailed probability p<0,0001. Decrease of pain sensation was recorded in 91.7% of patients. The improvement was more than 50% in 80% of the patients. (See FIG. 7.)

Walking distance and time to claudication at 3 miles per hour was tested using a treadmill (using median values and 95% confidence intervals). The distance to claudication before and after treatment was determined to be: Before the protocol, the initial Median was 100 CI (50 to 200) and at the end of 6 weeks the final Median was 535 CI (382.947 to 800). The difference was statistically significant. Wilcoxon test (paired samples) Two-tailed probability p<0.0001. (See FIG. 8.)

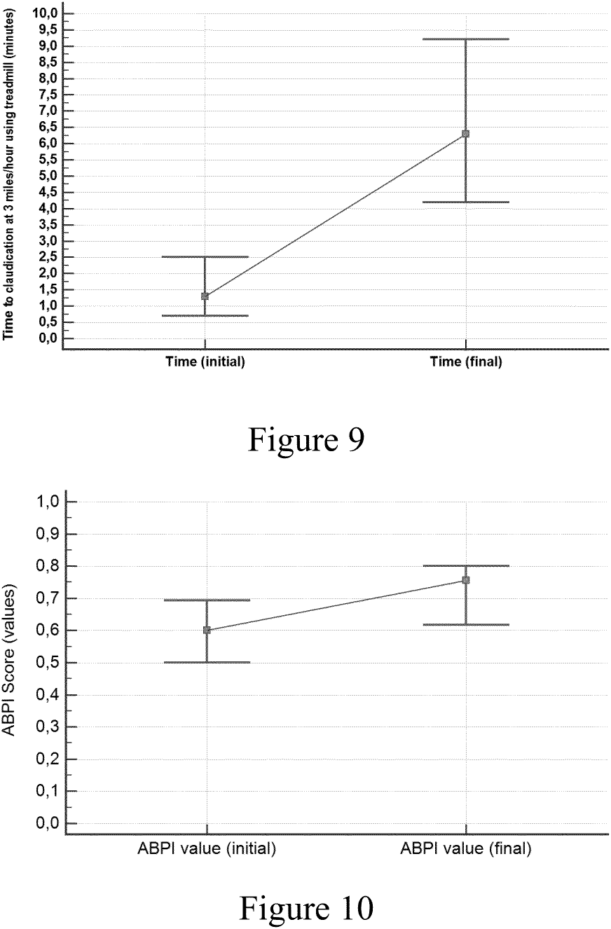

Time to claudication before hydration: The time to claudication was determined using a treadmil (using median values and 95% confidence interval) before and after treatment. Before treatment, the initial Median was 1.3 minutes (0.7 to 2.500) minutes, and after hydration the Median was 6.3 minutes (4.195 to 9.209) minutes. (See FIG. 9.) The difference was statistically significant, Wilcoxon test (paired samples) Two-tailed probability p<0.0001.

Ankle brachial pressure index: Ankle /brachial index was determined before and after treatment (using median values and 95% confidence intervals). The initial Median index before treatment was 0.6 (0.50 to 0.69), and the Median index after 6 weeks of treatment 0.75 (0.62 to 0.80). (See FIG. 10.) The difference was statistically significant. Wilcoxon test (paired samples) Two-tailed probability p<0,0001.

The thermographic images of FIGS. 3 and 4 correspond to patient 15.

In summary:
Previous intake of water: 1000 ml; Final intake of water: 3000 ml
Initial ankle/brachial index: 0.77; Final ankle/brachial index: 0.83
Distance of claudication (initial): 300 mts; Distance to claudication (final):
6000 mts
Initial pain intensity sensation: 6; Final pain intensity sensation: 0

Prior to this study, most patients presenting with ischemia of the legs had not achieved appropriate hydration. Results of this study using ankle/brachial index, cutaneous temperature, distance and time to claudication using the treadmill and subjective quantitative assessment of pain demonstrated differences statistically significant in patients who drank more than 2.5 liters a day. Patients, after being subjected to controlled hydration, improved dramatically and were excluded from the waiting list to have an endovascular intervention or surgery.

The clinical improvement occurred in patients regardless of the level of arterial occlusions detected by color duplex scans. The fact that dehydration is often seen in elderly population and could cause aggravation of function of all organs is well recognized by the medical community. Dehydration is common in elderly patients, one of every ten admissions to American hospitals are because of dehydration. Dehydration can occur because fluid losses or decreased intake. Elderly people drink less fluids because the sensation of thirst decreases. This decrease of thirst sensation can occur because depletion in dopamine level and increased level of plasma Atrial Natriuretic Peptide (ANP). The body water composition decreases from 70 to 55% because of the decrease in muscles and an increase in fatty tissue, renal function is impaired with less capacity to concentrate, increasing urine formation. Kidneys are also less reactive to ADH and have a lower ability to regulate sodium excretion. Lastly, iatrogenic factors can aggravate dehydration (Laxatives, diuretics or angiotensin converting enzyme convertors).

Early diagnosis is sometimes difficult because the classical physical signs of dehydration may be absent or misleading in an older patient. What it is rarely considered is the degree of aggravation of lower extremities intermittent claudication, dehydration could cause.

The degree of response to dinking the appropriate amount of fluids in a controlled way every day is significant. Anecdotally, even patients with ulcers in the toes and ankle, reversed the lesions just drinking 3 liters of water a day, the same good result was obtained in a patient who had one or two episodes of syncope caused by non-correctable cerebrovascular disease. Since requiring the patients to drink between 2.5 to 3 liters of water a day, less patients needed endovascular or open interventions, saving money and patient suffering.

The results seem to indicate that 5% of the body water resides intravascular, dehydration decreases perfusion because of the decreased blood volume. Interstitial water also decreases the exchange of nutrients, products of catabolism, and blood gases between the capillaries and the cells.

Most of elderly patients affected by peripheral arterial disease studied in this trial were dehydrated. Proper hydration improved clinical symptoms and objective assessment of peripheral perfusion. A majority of patients had an increase of ankle/brachial Index, distance and time of intermittent claudication using a treadmill and an increase in cutaneous temperature of the feet. The study suggests that proper hydration should be included in the armamentarium of the angiologist and vascular surgeon.

While several embodiments have been described, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus, the scope of the embodiments should be determined by the appended claims and their legal equivalents, rather than by the examples given.

Persons skilled in the art will understand that the compositions and methods specifically described herein are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly described, except as indicated by the appended claims.

What is claimed is:

1. A system for administering one or more hydration compositions to a patient, the system comprising:
a hydration composition delivery element
a sensor operatively coupled to the hydration composition delivery element and configured to determine one or more of the intake of fluids, the amount of fluid discharged, the activity level of the patient, or level of hydration of the patient based on the level of fluid in the hydration composition delivery element; and
a monitoring system which, based on a signal received from the sensor, provides an indication of one or more of the need to intake fluid, the identity of a specific hydration composition of a plurality of hydration compositions to be consumed or an amount of a hydration composition to be consumed.

2. The system of claim 1, wherein the monitoring system is configured to determine an individual’s level of electrolytes.

3. The system of claim 1, wherein the monitoring system is configured to determine an individual’s level of blood sugar.

4. The system of claim 1, wherein the monitoring system is configured to determine an individual’s level of tissue perfusion.

5. The system of claim 1, wherein the monitoring system is configured to determine an individual’s oxygen saturation.

6. The system of claim 1, wherein the monitoring system is configured to determine anticipated periods of dehydration throughout a day.

7. The system of claim 1, wherein the monitoring system is configured to track fluid consumption and measure the hydration of the user through an oral, infrared, ultrasound, optical or other sensor.

8. A method of administering one or more hydration compositions to a patient, the method comprising:
detecting a hydration-indicative parameter of the patient, the hydration-indicative parameter being selected from a group consisting essentially of: an amount of fluid intake, an amount of fluid discharged, an activity level of the patient, and a level of hydration of the patient;
generating a signal indicative of the hydration-indicative parameter;
transmitting the signal to a monitoring system; and
based on the signal received from the sensor, providing an indication of a need to intake fluid, an identity of a specific hydration composition of a plurality of hydration compositions to be consumed or an amount of a hydration composition to be consumed.

9. The method of claim 8, further comprising providing a sensor operatively coupled to a hydration composition delivery element, the sensor configured to determine one or more of the amount of fluid intake, the amount of fluid discharged, the activity level of the patient, or the level of hydration of the patient based on the level of fluid in the hydration composition delivery element.

10. The method of claim 9, wherein the sensor is coupled to a flexible package, a bottle or other element capability of holding fluid.

11. The method of claim 9 further comprising displaying the need to intake fluid.

12. The method claim 9 further comprising displaying an identity of a specific hydration composition of a plurality of hydration compositions to be consumed.

13. The method of claim 9 further comprising displaying an amount of a hydration composition to be consumed.
